SEQUENCE LISTING (12 sequences)

SEQ ID NO: 1 - DNA, 900 bp, Artificial Sequence
  Description of Artificial SequenceThe DNA sequence encodes the rSOWgp58 which is use as an antigen
atgggcagcagccatcatcatcatcatcacagcagcggcctggtgccgcgcggcagccatatggctagcatgactggtggacagcaaatgggtcgggatccgaattctgcagattacagttattgcgatacctatggttgtgacggcccaatggagccaaaaacccctaagccaacagattgttatggtgactgcgaggatgggtatgattactcccctccacctcctcccaagaagtatggtgactgtgatgattatgatgggtattgtgatggtccctcaaaaacctcaatgaagccagaaccccctaagccaacagattgttatggtgactgcgaggatgggtatgattactcccctccacctcctcccaagaagtatggtgactgtgatgattatgacgggtattgtgatggtccctcaaaaacctcaatgaagccagaaccccctaagccaacagattgttatggtgactgtgaggatgggtatgattactcccctccaccacctcccaagaagtatggtgactgtgatgattatgatgggtattgtgatggcccctcaaaaacttcaataaagccagaaccccctaagccaacagattgttatggtgactgtgaggatgggtatgattactcccctccaccacctcccaagaagtatggcgactgtgattatgatgatgggtattgcgacggttcccctccacctaaggaaacaaagaattatggctaccattatgccagagcccatgacaaggcagcttcctctggcagcacagaccggccagcttctacaccagttcatacgccaactcagtttgagggggctgcaggaatcttgcagccgcggggtatctctttcgtcgcccttgctatcgcagctgcattctatgtttga

SEQ ID NO: 2 - protein, 299 aa, Artificial Sequence
  Description of Artificial SequenceThe amino acid sequence of rSOWgp58 which includes 39 aa from the vector and 266 aa from SOwgp58 of Coccidioides immitis
MGSSHHHHHHSSGLVPRGSHMASMTGGQQMGRDPNSADYSYCDTYGCDGPMEPKTPKPTDCYGDCEDGYDYSPPPPPKKYGDCDDYDGYCDGPSKTSMKPEPPKPTDCYGDCEDGYDYSPPPPPKKYGDCDDYDGYCDGPSKTSMKPEPPKPTDCYGDCEDGYDYSPPPPPKKYGDCDDYDGYCDGPSKTSIKPEPPKPTDCYGDCEDGYDYSPPPPPKKYGDCDYDDGYCDGSPPPKETKNYGYHYARAHDKAASSGSTDRPASTPVHTPTQFEGAAGILQPRGISFVALAIAAAFYV

SEQ ID NO: 3 - DNA, 1244 bp, Coccidioides immitis Silveria
aagagaactgtatgcacatcttcttctgatctaattcgggtttactttccggtgcaaaactttcgccatgaaaatctttcgcataatcatcatctcagccatagcagcttgtccctttgccatcggctacgagtatccgccacatgattgccaatcttacggtgatgattatggaaattgtaaaggagagaagccttctgccacgcccagtcattatgatgaatatggatataaaatgaggaaacgaggtgctacgagccacaaagagcacagttattgcgatacctatggttgtgacggcccaatggagccaaaaacccctaagccaacagattgttatggtgactgcgaggatgggtatgattactcccctccacctcctcccaagaagtatggtgactgtgatgattatgatgggtattgtgatggtccctcaaaaacctcaatgaagccagaaccccctaagccaacagattgttatggtgactgcgaggatgggtatgattactcccctccacctcctcccaagaagtatggtgactgtgatgattatgacgggtattgtgatggtccctcaaaaacctcaatgaagccagaaccccctaagccaacagattgttatggtgactgtgaggatgggtatgattactcccctccaccacctcccaagaagtatggtgactgtgatgattatgatgggtattgtgatggcccctcaaaaacttcaataaagccagaaccccctaagccaacagattgttatggtgactgtgaggatgggtatgattactcccctccaccacctcccaagaagtatggcgactgtgattatgatgatgggtattgcgacggttcccctccacctaaggaaacaaagaattatggctaccattatgccagagcccatgacaaggcagcttcctctggcagcacagaccggccagcttctacaccagttcatacgccaactcagtttgagggggctgcaggaatcttgcagccgcggggtatctctttcgtcgcccttgctatcgcagctgcattctatgtttgatttcagagaatttcttttcccctttttgttgtttggcagtagatgtcttcagctttagagcgggaacaagttgggtagatggatttgattttgaaaatccaagtctctccttacaaacagccagcaatcctactttcctttcttcgaatacagccgcgcaaaatattagtcacgggctcattgtctcctc

SEQ ID NO: 4 - protein, 328 aa, Coccidioides immitis Silveria
MKIFRIIIISAIAACPFAIGYEYPPHDCQSYGDDYGNCKGEKPSATPSHYDEYGYKMRKRGATSHKEHSYCDTYGCDGPMEPKTPKPTDCYGDCEDGYDYSPPPPPKKYGDCDDYDGYCDGPSKTSMKPEPPKPTDCYGDCEDGYDYSPPPPPKKYGDCDDYDGYCDGPSKTSMKPEPPKPTDCYGDCEDGYDYSPPPPPKKYGDCDDYDGYCDGPSKTSIKPEPPKPTDCYGDCEDGYDYSPPPPPKKYGDCDYDDGYCDGSPPPKETKNYGYHYARAHDKAASSGSTDRPASTPVHTPTQFEGAAGILQPRGISFVALAIAAAFYV

SEQ ID NO: 5 - protein, 15 aa, Coccidioides immitis C735
GATSHKEHSYCDTYG

SEQ ID NO: 6 - protein, 10 aa, Coccidioides immitis C634
GATSHKEHSY

SEQ ID NO: 7 - protein, 10 aa, Coccidioides immitis Silveria
GATSHKEHSY

SEQ ID NO: 8 - protein, 14 aa, Coccidioides immitis C735
TPKPTDCYCDCEDG

SEQ ID NO: 9 - DNA, 1504 bp, Coccidioides immitis C634
aattcgggtttactttccggtgcaaaaactttcgccatgaaaatctttcgcataatcatcatctcagccatagcagcttgtccctttgccatcggctacgagtatccgccacatgattgccaatcttacggtgatgattatggaaattgtaaaggagagaagccttctgccacgcccagtcattatgatgaatatggatataaaatgaggaaacgaggtgctacgagccacaaagagcacagttattgcgatacctatggttgcgacggcccaatggagccaaaaacccctaagccaacagattgttatggtgactgcgaggatgggtatgattactcccctccaccacctcccaagaagtatggtgactgtgatgattatgatgggtattgtgatggcccctcaaaaacttcaataaagccagaaccccctaagccaacagattgttatggtgactgcgaggatgggtatgattactcccctccaccacctcccaagaagtatggtgactgtgatgattatgatgggtattgtgatggcccctcaaaaacttcaataaagccagaaccccctaagccaacagattgttatggtgactgcaaggatgggtatgattactcccctccacctcctcccaagaagtatggtgactgtgatgattatgacgggtattgtgatggtccctcgaaaacctcaatgaagccagaaccccctaagccaacagattgttatggtgactgcgaggatgggtatgattactcccctccaccacctcccaagaagtatggtgactgtgattatgatgatgggtattgtgatggcccctcgaaaacctcaataaagccagaaccccctaagccaacagattgttatggtgactgtgaggatgggtatgattactcccctccaccacctctcaagaagtatggtgactgtgatgattatgatgggtattgtgatggcccctcaaaaacttcaataaagccagaaccccctaagccaacagattgttatggtgactgtgaggatgggtatgattactcccctccaccacctcccaagaagtatggtgactgtgattatgatgatgggtattgcgacggttcccctccacctaaggaaacaaagaattatggctaccattatgccagagcccatgacaaggcagcttcctctggcagcacagaccggccagcttctacaccagttcatacgccaactcagtttgagggagctgcaggaatcttgcagccgcggggtatctctttcgtcgcccttgctatcgcagctgcattctatgtttgatttcagagaatttcttttcccctttttgttgtttggcagtagatgtcttcagctttagagcgggaacaagttgggtagatggatttgattttgaaaatccaagtctctccttacaaacagccagcaatcctactttcctttcttcgaatacagccgcgcaaaatattagtcacgggctcattgtctcctcaaaaaaaaa

SEQ ID NO: 10 - protein, 375 aa, Coccidioides immitis C634
MKIFRIIIISAIAACPFAIGYEYPPHDCQSYGDDYGNCKGEKPSATPSHYDEYGYKMRKRGATSHKEHSYCDTYGCDGPMEPKPPKPTDCYGDCEDGYDYSPPPPPKKYGDCDDYDGYCDGPSKTSMKPEPPKPTDCYGDCEDGYDYSPPPPPKKYGDCDYDDGYCDGPSKTSMKPEPPKPTDCYGDCEDGYDYSPPPPPKKYGDCDDYDGYCDGPSKTSMKPEPPKPTDCYGDCEDGYDYSPPPPPKKYGDCDDYDGYCDGPSKTSMKPEPPKPTDCYGDCEDGYDYSPPPPPKKYGDCDYDDGYCDGSPPPKETKNYGYHYARAHDKAVSSGSTDRPASTPVHTPTQFEGAAGILQPRGISFVALAIAAAFYV

SEQ ID NO: 11 - DNA, 1563 bp, Coccidioides immitis C735
acatcttcttctgatctaattcgggtttactttccggtgcaaaaactttcgccatgaaaatctttcgcataatcatcatctcagccatagcagcttgtccctttgccatcggctacgagtatccgccacatgattgccaatcttacggtgatgattatggaaattgtaaaggagagaagccttctgccacgcccagtcattatgatgaatatggatataaaatgaggaaacgaggtgctacgagccacaaagagcacagttattgcgatacctatggttgcgacggcccaatggagccaaaaacccctaagccaacagattgttatggtgactgcgaggatgggtatgattactcccctccaccacctcccaagaagtatggtgactgtgatgattatgatgggtattgtgatggcccctcaaaaacttcaataaagccagaaccccctaagccaacagattgttatggtgactgcgaggatgggtatgattactcccctccaccacctcccaagaagtatggtgactgtgatgattatgatgggtattgtgatggcccctcaaaaacttcaataaagccagaaccccctaagccaacagattgttatggtgactgcaaggatgggtatgattactcccctccacctcctcccaagaagtatggtgactgtgatgattatgacgggtattgtgatggtccctcgaaaacctcaatgaagccagaaccccctaagccaacagattggtatggtgactgcgaggatgggtatgattactcccctccaccacctcccaagaagtatggtgactgtgattatgatgatgggtattgtgatggcccctcgaaaacctcaataaagccagaaccccctaagccaacagattgttatggtgactgtgaggatgggtatgattactcccctccaccacctctcaagaagtatggtgactgtgatgattatgatgggtattgtgatggcccctcaaaaacttcaataaagccagaaccccctaagccaacagattgttatggtgactgtgaggatgggtatgattactcccctccaccacctcccaagaagtatggtgactgtgattatgatgatgggtattgcgacggttcccctccacctaaggaaacaaagaattatggctaccattatgccagagcccatgacaaggcagcttcctctggcagcacagaccggccagcttctacaccagttcatacgccaactcagtttgagggagctgcaggaatcttgcagccgcggggtatctctttcgtcgcccttgctatcgcagctgcattctatgtttgatttcagagaatttcttttcccctttttgttgtttggcagtagatgtcttcagctttagagcgggaacaagttgggtagatggatttgattttgaaaatccaagtctctccttacaaacagccagcaatcctactttcctttcttcgaatacagccgcgcaaaatattagtcacgggctcattgtctcctcaaaatgttcattttcaataatgtagagccaatgatagcacttcttaagtct

SEQ ID NO: 12 - protein, 422 aa, Coccidioides immitis C735
MKIFRIIIISAIAACPFAIGYEYPPHDCQSYGDDYGNCKGEKPSATPSHYDEYGYKMRKRGATSHKEHSYCDTYGCDGPMEPKTPKPTDCYGDCEDGYDYSPPPPPKKYGDCDDYDGYCDGPSKTSIKPEPPKPTDCYGDCEDGYDYSPPPPPKKYGDCDDYDGYCDGPSKTSIKPEPPKPTDCYGDCKDGYDYSPPPPPKKYGDCDDYDGYCDGPSKTSMKPEPPKPTDWYGDCEDGYDYSPPPPPKKYGDCDYDDGYCDGPSKTSIKPEPPKPTDCYGDCEDGYDYSPPPPLKKYGDCDDYDGYCDGPSKTSIKPEPPKPTDCYGDCEDGYDYSPPPPPKKYGDCDYDDGYCDGSPPPKETKNYGYHYARAHDKAASSGSTDRPASTPVHTPTQFEGAAGILQPRGISFVALAIAAAFYV

(19) United States
(12) Patent Application Publication
(10) Pub. No.: US 2002/0106380 A1
(43) Pub. Date: Aug. 8, 2002

(54) rSOWgp58 protein and its use as an antigen

(75) Inventors: Chiung-Yu Hung, Toledo, OH (US); Garry T. Cole, Toledo, OH (US)

Correspondence Address: EMCH, SCHAFFER, SCHAUB & PORCELLO CO, P O BOX 916, ONE SEAGATE SUITE 1980, TOLEDO, OH, 43697

(73) Assignee: Medical College of Ohio

(21) Appl. No.: 09/850,677

(22) Filed: May 7, 2001

Related U.S. Application Data
(60) Provisional application No. 60/202,754, filed on May 8, 2000.

(51) Int. Cl. A61K 39/002; G01N 33/53; G01N 33/569; C07K 14/44

(52) U.S. Cl. 424/191.1; 435/7.22; 530/395

(57) ABSTRACT

The present invention provides bacterial plasymids and recombinant SOW proteins that are useful as antigens for serodiagnosis of coccidiomycosis.

### RELATED APPLICATION

The present application is related to U.S. Provisional Application Serial No. 60/202,754 filed May 8, 2000.

### BACKGROUND OF THE INVENTION

Coccidioides immitis is the causative agent of a human respiratory disease known as San Joaquin Valley fever or coccidioidomycosis. The pathogen is a diphasic fungus that produces mycelia and air-dispersed spores (arthroconidia) when grown on simple glucose-yeast extract (GYE) agar medium and gives rise to endosporulating parasitic cells (spherules) when arthroconidia infect mammalian lung tissue (9). The parasitic cycle of C. immitis is reproduced in vitro by growth in a defined glucose-salts medium at 39° C. with the addition of 20% CO₂ (24). Although it is not certain that in vivo and in vitro presentations of cell surface antigens by the parasitic phase of the fungal pathogen are identical, morphological details of C. immitis grown under these two conditions appear to be the same (35). Evidence has also been presented by numerous studies that purified, wall-associated antigens of C. immitis isolated from in vitro-grown cells are recognized by sera from patients with coccidioidal infection (10, 18, 23, 28, 29).

We suggest that the identification of immunodominant C. immitis antigens which elicit potent responses of both the humoral and cellular immune systems is of particular interest because their presentation in vivo may profoundly influence the course of disease. Protection against coccidioidomycosis and related fungal respiratory infections (e.g., blastomycosis and paracoccidioidomycosis) is correlated with strong delayed-type hypersensitivity response in humans (19, 33, 38). Activation of the T helper 1 (Th1) rather than the Th2 subset of T cells appears to be pivotal for immunoprotection against C. immitis infection (25, 26). Klein and Newman have identified a major immunoreactive, 120-kDa cell wall glycoprotein which is expressed at the surface of yeast of the respiratory pathogen Blastomyces dermatitidis, and it appears to play a key role in the pathogenesis of the fungus (22). The antigen (Wl-1) has been shown to be strongly reactive in both humoral and cellular immunoassays, and it is expressed by all virulent isolates that have so far been examined. Purified WI-1 has been shown to contain epitopes which mediate attachment of the parasitic cells to human macrophages, and it elicits a potent B-cell response. A 43-kDa glycoprotein (gp43) presented at the surface of infectious yeast of Paracoccidioides brasiliensis has been shown to elicit both a strong humoral response and delayed-type hypersensitivity reaction in humans (33). The immunodominance of gp43 as a cell surface antigen is based on its recognition by virtually 100% of the sera from patients with confirmed paracoccidioidomycosis. The gp43 antigen is a receptor for laminin-1 and binds to the surface of macrophages (1, 37). High anti-gp43 titers in infected patients have been suggested to correlate with cellular immune hyporesponsiveness to a crude cell wall extract of P. brasiliensis (3, 4). Both WI-1 and gp43 appear to have the potential, as parasitic cell surface-presented immunodominant antigens, to modulate levels of cellular and humoral responses of the host and thereby influence the outcome of the mycosis.

We previously described a membranous spherule outer wall (SOW) fraction produced by parasitic-phase cultures of C. immitis (8, 11). SOW was characterized by high levels of reactivity in both cellular and humoral immunoassays. The SOW fraction isolated from liquid cultures of C. immitis spherules was shown to react with patient anti-Coccidioides antibody based on immunofluorescence and immunodiffusion-tube precipitin assays (11). Serologically reactive components of the SOW were extracted from the isolated wall fraction by using the nonionic detergent N-octyl-β-D-glucopyranoside (OG). Compositional analysis of the dialyzed detergent extract by protein electrophoresis suggested that the solubilized fraction of isolate C735 contained two major polypeptides (11). Two-dimensional immunoelectrophoresis (2D-IEP) with burro antiserum raised to C. immitis mycelium-derived coccidioidin was used to examine the antigenic composition of the OG-soluble fraction of SOW (11). Although patient sera used in immuno-blots recognized the dominant polypeptide of the SOW extract, the 2D-IEP failed to show the presence of a major precipitin arc. The OG-soluble fraction was recognized by sera from all patients with coccidioidomycosis who were tested in the enzyme-linked immunosorbent assay (ELISA) but not by sera of any of the control patients (11). The crude SOW and OG-soluble fraction were both shown to be potent elicitors of murine immune T-cell proliferation, as demonstrated by results of in vitro cellular immunoassays (1 1). More recently, the crude SOW fraction of C. immitis was used to immunize BALB/c mice against a lethal challenge of the pathogen (20). Subcutaneous immunization with the isolated wall material resulted in a 100-fold decrease in the number of CFU of C. immitis per lung compared to infected lungs of control mice.

### SUMMARY OF THE INVENTION

The present invention provides the isolation and characterization of the immunodominant glycoprotein component of SOW (SOWgp) which is responsible for the mixed humoral and cellular immune responses to the spherule wall fraction of C. immitis.

Multinucleate parasitic cells (spherules) of Coccidioides immitis isolates produce a membranous outer wall component (SOW) in vitro which has been reported to be reactive with antibody from patients with coccidioidal infection, elicit a potent proliferative response of murine immune T cells, and have immunoprotective capacity in a murine model of coccidioidomycosis. To identify the antigenic components of SOW, the crude wall material was first subjected to Triton X-114 extraction, and a water-soluble fraction derived from this treatment was examined for protein composition and reactivity in humoral and cellular immunoassays. Protein electrophoresis revealed that the aqueous fraction of three different isolates of C. immitis each contained one or two major glycoproteins (SOWgps), distinguished by their molecular sizes, which ranged from 58 to 82 kDa. The SOWgps, however, showed identical N-terminal amino acid sequences, and each was recognized by sera from patients with C. immitis infection. Antibody raised against the purified 58-kDa glycoprotein (SOWgp58) of the Silveira isolate was used for Western Blot and immunolocalization analyses. Expression of SOWgp was shown to be parasitic phase specific, and the antigen was localized to the membranous SOW. The water-soluble fraction of SOW and the purified SOWgp58 were tested for the ability to stimulate proliferation of human peripheral monocytic cells (PBMC). The latter were obtained from healthy volunteers with positive skin test reaction to spherulin, a parasitic-phase antigen of C. immitis, and from volunteers who showed no skin test reaction to the same antigen. The SOW preparations stimulated proliferation of PBMC from skin test-positive but not skin test-negative donors, and the activated cells secreted gamma interferon, which is indicative of a T helper 1 pathway of immune response. SOWgp is a major parasitic cell surface-expressed antigen that elicits both humoral and cellular immune responses in patients with coccidioidal infection.

The Seq. No. 1 provides the DNA sequence of the Escherichia coli (bacterium)-expressed SOWgp58 gene which contains 117 base pairs (bp) of the bacterial vector sequence and 783 bp of the SOWgp58 gene of Coccidioides immitis.

The Seq. No. 2 provides the amino acid sequence of the recombinant SOWgp58 (rSOWgp58), which includes 39 amino acid residues (aa) of the vector and 260 aa of SOWgp58 of Coccidioides immitis.

The Seq. No. 3 provides the DNA sequence of the SOWgp58 gene of the Silveria isolate of Coccidioides immitis.

The Seq. No. 4 provides the amino acid sequence of SOWgp58 of the Silveria isolate of Coccidioides immitis.

The Seq. No. 5 provides the amino acid sequence of a peptide fragment of SOWgp82 isolated from the C735 isolated of Coccidioides immitis.

The Seq. No. 6 provides the amino acid sequence of a peptide fragment of SOWgp66 isolated from the C634 isolate of Coccidioides immitis.

The Seq. No. 7 provides the amino acid sequence of a peptide fragment of SOWgp58 isolated from the Silveria isolate of Coccidioides immitis.

The Seq. No. 8 provides the amino acid sequence of a peptide fragment of SOWgp82 isolated from the C735 isolate of Coccidioides immitis.

The Seq. No. 9 provides the DNA sequence of the SOWgp66 gene of the C634 isolate of Coccidioides immitis.

The Seq. ID No. 10 provides the amino acid sequence of SOWgp66 of the C634 isolate of Coccidioides immitis.

The Seq. ID No. 11 provides the DNA sequence of the SOWgp82 gene of the C735 isolate of Coccidioides immitis.

The Seq. ID No. 12 provides the amino acid sequence of SOWgp82 of the C735 isolate of Coccidioides immitis.

The invention also includes nucleic acids that hybridize under stringent hybridization conditions (as defined herein) to all or a portion of the nucleotide and/or amino acid sequence represented by the SEQ ID Nos. disclosed herein or their complements. The hybridizing portion of the hybridizing nucleic acids is typically at least 15 (e.g., 20, 25, 30, or 50) nucleotides in length. The hybridizing portion of the hybridizing nucleic acid is at least 80%, e.g., at least 95%, or at least 98%, identical to the sequence of a portion or all of a nucleic acid encoding the peptide, or its complement. Hybridizing nucleic acids of the type described herein can be used, for example, as a cloning probe, a primer (e.g., a PCR primer), or a diagnostic probe. Hybridization of the oligonucleotide probe to a nucleic acid sample typically is performed under stringent conditions. Nucleic acid duplex or hybrid stability is expressed as the melting temperature of Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE).

Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the target nucleic acid. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecules but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (c) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

A conservative amino acid substitution is a replacement of one or more amino acid residues with at least one different residue (in certain embodiments from about 0 to about 20) having similar physicochemical characteristics, such as size, change, and polarity/nonpolarity.

The novel sequences disclosed herein are useful as antigens for the serodiagnosis of coccidiodomysis and can be incorporated into a suitable kit for use as a diagnostic test for coccidiodomysis.

### DESCRIPTION OF THE FIGURES

FIG. 1 is a summary of the protocol used to extract SOWgps from C. immitis isolates. r.t., room temperature. Asterisks denote fractions which were examined by SDS-PAGE in FIG. 2.

FIG. 2 shows a (A) SDS-PAGE (10% polyacrylamide) silver-stained gel separations of aqueous fraction of the TX114-derived extract, SDS-solubilized fraction, and NaOH-solubilized fraction of C. immitis isolate C735; (B) silver-stained gel separations of aqueous fractions of isolates C634, C735, and Silveira (Sil.) as above. Std., size standards; Aq.Fr., aqueous fraction.

FIG. 3 shows a (A) SDS-PAGE (10% polyacrylamide) separation of purified SOWgps of three isolates of C. immitis; (B) SDS-PAGE (12% polyacrylamide) separation of major peptide components of the Lys-C-digested 82- and 60-kDa fractions of the C735 SOWgp isolate. Std., size standards.

FIG. 4 shows SDS-PAGE (10% polyacrylamide) separation, PAS reagent stain, and Western blot (W.B.) of SOWgp of three C. immitis isolates. Western blotting was performed with sera from patients with confirmed coccidioidomycosis. Std., size standards; Sil., Silveira.

FIG. 5 shows (A) Coomassie blue-stained SDS-PAGE (12% polyacrylamide) separation of aqueous fraction of the TX114-derived SOW extract of three C. immitis isolates; (B and C) Western blot analysis using rabbit antiserum raised against purified, native AgCS and goat antiserum raised against purified, native PRAG2, respectively. The Western blots in panels B and C also include electrotransferred rAgCS and rPRAg2 as positive controls. Std., size standards; Sil., Silveira.

FIG. 6 shows (A) Light micrographs of C. immitis vegetative mycelial phase (M) harvested from culture at 4 days and parasitic cells isolated from cultures at 1.5 days (spherule initials; S1), 3 days (segmented spherules; S2), and 5 days (endosporulating spherules; S3). Bars for M to S3 represent 100, 50, 20, and 40 μm, respectively (A). (B and C) SDS-PAGE (10% polyacrylamide) separation (B) and corresponding Western blot (C) of total cell homogenates of the mycelial and parasitic cells (M, S1, S2, and S3) described above. Equal amounts of protein were applied to each lane in panel B. Murine antiserum raise against the purified 58-kDa SOWgp of the Silveira isolate (SOWgp58) was used for the Western blot in panel C. Std., size standards.

FIG. 7 shows (A to C) Immunofluorescence light micrographs of spherules reacted with anti-SOWgp58 antiserum followed by secondary antibody-FITC conjugate, showing discontinuous cell surface label. Note that the larger, more mature spherule in panel A is more intensely labeled than the young spherule. The arrows in panel C indicate the fluorescence-labeled membranous layers of the SOW, which are released into the culture media. The bright-field (D) and matching immunofluorescence micrographs (E) of spherules reacted with the secondary antibody-FITC conjugate alone or controls. The latter shows no labeling of the cells.

FIG. 8 shows the results of ELISA with control patient sera and sera from patients with confirmed mycotic infections (blastomycosis, histoplasmosis, or coccidioidomycosis) adsorbed to the 82-kDa SOWgp of isolate C735 bound to wells of microdilution plates (10 ng/well). Goat anti-human IgG (H+L) conjugated to peroxidase was used for the detection of adsorbed antibody. The value for the mean OD of the control serum sample plus twice the standard deviation (0.11) is shown as a dashed line. All OD values for the test sera plotted above the dashed line are considered to show a positive reaction with the SOWgp82.

FIG. 9 shows the comparison of seroreactivity of representative patient serum (no. 6 in Table 2) in an inhibition ELISA with SOWgp58 or −82 bound to wells of microtiter plates. Different concentrations of homologous or heterologous SOWgp were used to preincubate with patient serum and inhibit antibody reaction with immobilized antigen (Ag).

FIG. 10 shows the proliferative responses of PBMC isolated from spherulin skin test-positive and -negative, healthy human donors to the aqueous fraction of the TX114 extract of SOW of the Silveira isolate (A) and purified SOWgp58 of the same isolate (B). Data are presented as means ± standard errors of the means of the stimulation index (SI) (counts per minute of wells of microdilution plates which contain the antigen divided by counts per minute of control wells without antigen).

The P values determined by the Mann-Whitney U test are shown for comparison of proliferation results between the two donor groups.

### DETAILED DESCRIPTION OF THE INVENTION

The release of membranous sheets of wall material from the surface of C. immitis spherules is a common feature of isolates of this fungal pathogen. Transmission electron microscopic studies of the SOW fraction show that the secreted material is osmiophilic, which is indicative of the presence of lipids, and cross sections of the isolated SOW revealed a tripartite structure, reminiscent of biomembranes (8). Early investigators have reported that the walls of mature spherules are rich in “lipid complexes” (36). The chemical composition of the whole spherule wall (9) is distinct from that of the isolated SOW. The neutral carbohydrate, nitrogen, and lipid content of the SOW are 16.7, 64.7, and 10.5%, respectively, of the total dry weight of wall material (unpublished data). These estimates are based on analytical methods previously reported (9). Frey and Drutz (14) described an extracellular matrix produced by parasitic cells of C. immitis which they suggested may impede contact between host polymorphonuclear neutrophils and spherules. Parasitic cells grown on Converse agar are commonly clustered due to the confluence of the SOW material which binds the cells together (8). A similar clustering of parasitic cells has been shown to occur in vivo (6), and this could further compromise the ability of host defense cells to clear infections of C. immitis. Under in vivo growth conditions, the SOW layer may also act as a reservoir of antigens which are transported to the cell wall and entrapped by what is an organic polymer matrix (biofilm-like layer [27, 34]) formed at the surface of clusters of parasitic cells. The presence of such a matrix explains how the hydrophilic glycoproteins (SOWgps), identified by SDS-PAGE as the dominant proteinaceous components of the SOW, remains associated with the membranous wall fraction. The SOWgps are either released from the matrix or presented at its surface, since C. immitis-infected patients uniformly recognize the glycoproteins. It is also believed that the SOW matrix of certain isolates could at least partially mask the immunoreactive SOWgps, resulting in the range of antibody titers to the glycoproteins which was observed in this invention. Klein and coworkers (21) have proposed that α-(1 ,3)-glucan incorporation into the yeast wall of B. dermatitidis can mask the immunodominant WI-1 antigen and thereby control the amount of the glycoprotein released from the cell surface. Shedding of WI-1 was suggested to facilitate immune evasion by binding or consuming complement and antibody opsonins away from the yeast cell surface. Disruption of the WI-1 gene resulted in loss of virulence of B. dermatitidis in mice (5). The function of SOWgp in pathogen-host interactions is unknown. The SOWgp genes of the three isolates examined here have been cloned (C.-Y. Hung, J.-J. Yu, and G. T. Cole, Abstr. 99th Gen. Meet. Am. Soc. Microbiol. 1999, abstra. F-83, p. 312, 1999). Our recent development of a transformation system for C. immitis (U. Reichard, J.-J. Yu, K. R. Seshan, and G. T. Cole, Abstra. 99th Gen. Meet. Am. Soc. Microbiol. 1999, abstra. F-22, p. 299, 1999) provides the opportunity to generate SOWgp gene disruptants. The mutant strains are useful in the murine model of coccidioidomycosis to evaluate whether loss of expression of this dominant cell surface antigen alters virulence of the pathogen.

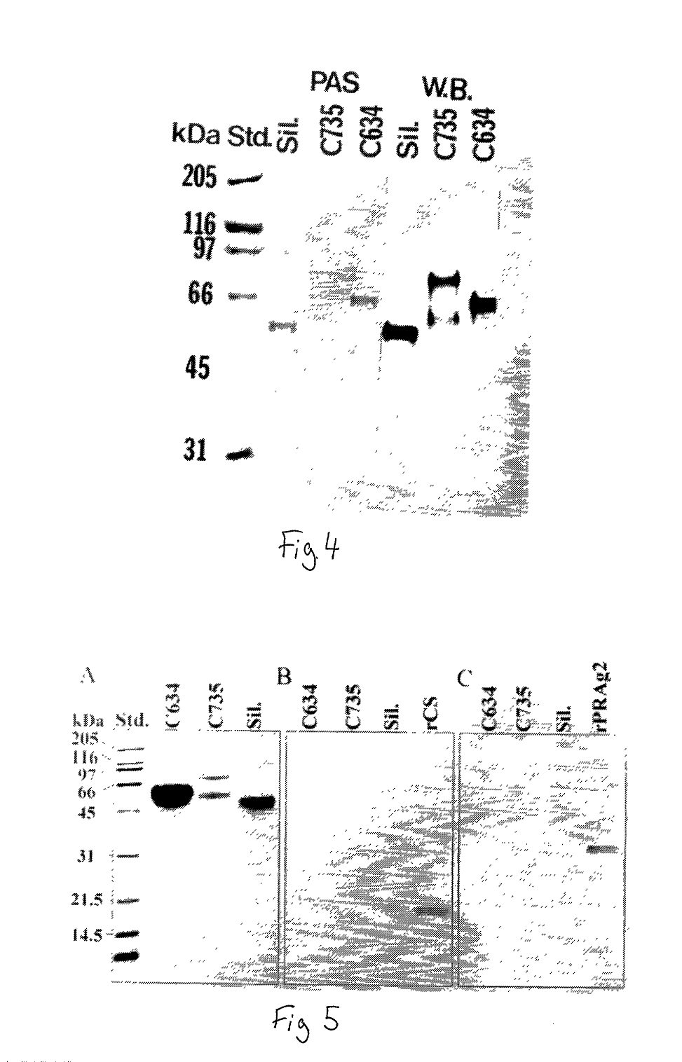

Although the N-terminal amino acid sequences of the SOWgps purified from three different isolates of C. immitis were identical, the difference in their molecular sizes raises questions about the structure of the mature proteins. In isolate C735, the SOWgp60 component (60-kDa glycoprotein) is a proteolytic product of the 82-kDa glycoprotein. Results of SDS-PAGE separations of the C735 preparations of the native SOW and peptide fingerprint comparison of Lys-C digestions of SOWgp82 and SOWgp60 support this conclusion. The difference in molecular sizes of the SOWgps of C735 (82 kDa), C634 (66 kDa), and the Silveira isolate (58 kDa) may be due to differences in length o the primary structure, posttranslational modification, or C-terminal truncation. All three SOWgps showed moderate to low levels of binding with PAS stain (FIG. 4), were resistant to endoglycosidase F digestion, and did not react with concanavalin A-peroxidase conjugate (not shown). These results suggest that glycosylation is not a major contributing factor to the molecular size of the SOWgps.

Expression of the SOWgps during growth of the pathogen appears to be restricted to stages of spherule-endospore formation. No SOW glycoprotein was detected by Western blot analysis of vegetative mycelial homogenates. During spherule development, it appeared that the highest amount of SOWgp was found in homogenates of endosporulating spherules. This was expected since the production of SOW increases with spherule maturation (8). Results of immunolocalization studies supported the finding that SOWgp expression is parasitic phase specific. Using anti-OSOWgp58 antibody, we demonstrate that the glycoprotein is present only at the surface of parasitic cells. This is the first phase-specific antigen of C. immitis so far reported. The discontinuous pattern of label at the spherule surface may be due to the sloughing of the membranous wall material, which has been reported to occur during growth of parasitic cells in liquid shake cultures (8). Another explanation is that the discontinuous pattern of fluorescent label is due to masking of the SOWgp by other components of the SOW matrix.

Previously reported studies of SOW antigenic composition were based on extraction of the spherule wall material with OG and analysis of the immunoreactivity of this extract by 2D-IEP using C. immitis mycelium-derived coccidioidin as the antigen reference system (8, 11). Since expression of SOWgps is parasitic phase specific, it is not surprising that we were unable to detect a prominent precipitin in the 2D-IEP gels (11). The detection of AgCS and PRAg2 as components of the soluble SOW fraction in our earlier studies (8, 11) was apparently the result of coextraction of these minor antigenic components together with SOWgp when OG was used as the solubilizing reagent. These antigens were not present in the aqueous phase of the TX114 extract reported here.

The purified SOWgps of the C735 and Silveira isolates show a ranged of seroreactivity in the ELISA with antibody from patients with confirmed coccidioidal infections. A similar range of OD values was reported for the ELISA of coccidioidomycosis patient serum reactivity with the crude SOW(11). The previously reported test sera which were incubated with the crude antigen were diluted 1:200 in blocking solution, while the patient sera in this study were diluted 1:2,000. The SOWgp is a major antigenic component of the SOW complex. The OD values for ELISAs using purified SOWgps from two distinct isolates of C. immitis show a high degree of correlation, which indicates that the difference in molecular sizes of the glycoproteins (SOWgp82 and SOWgp58) has little bearing on levels of patient seroreactivity. This is also supported by the results of the inhibition ELISA using a representative patient serum and SOWgps from two different isolates.

The titers of patient antibody to the CF antigen of C. immitis (30, 39) have no clear influence on the amplitude of OD values in the ELISAs with the purified SOWgps. Elevated anti-CF antibody titers (>1:16) of patients infected with C. immitis is prognostic of high risk or onset of disseminated coccidioidomycosis (30). Our observations of relatively high ELISA values and corresponding anti-CF titers of ≦1:16 for the same serum samples suggest that at least some patients mount a strong humoral response to the wall-associated glycoprotein early in the course of disease. Although all of the control patients with no evidence of mycotic disease showed consistently low OD values for seroreactivity with the SOWgps, some cross-reactivity was evident when sera from patients with confirmed blastomycosis and histoplasmosis were tested in the ELISA. This may be due to the presence of carbohydrate and/or protein homologs of SOWgp epitopes expressed by parasitic-phase cells of B. dermatitidis and H. capsulatum.

Results of the cellular immunoassays demonstrate that both the aqueous-soluble fraction of SOW separated from the TX114 extract of the spherule wall material and the purified SOWgp58 stimulated immune but not nonimmune human PBMC. The purified glycoprotein can elicit a proliferative response of monocytes of skin test-positive patients in vitro which is equal to or better than that observed in the presence of crude antigenic preparations (coccidioidin, spherulin, or toluene spherule lysate [2, 12]) when tested at similar concentrations (data not shown). SOWgp58 is the first purified antigen of C. immitis which has been shown to stimulate proliferation of human immune PBMC specifically. Isolation of sufficient amounts of purified SOWgp for cellular immunoassays is laborious and therefore problematic for comparison of SOWgps from multiple isolates. This obstacle can be overcome by using recombinant SOWgps. A possible pitfall, however, has that immunogenic proteins generated by recombinant methods would lose reactive epitopes present in the native antigen.

The culture supernatants of immune PBMC incubated with the aqueous-soluble fraction of the SOW detergent extract contain significantly higher amounts of INF-γ than the supernatants of nonimmune cells. The principal functions of INF-γ in vivo are the activation of macrophages and increased expression of major histocompatibility complex (MHC), which result in the stimulation of a Th1 pathway of host immune response (17). Based on the combined results of our immunoassays, it is shown herein that the parasitic cell surface-presented glycoprotein identified in this study is an immunodominant antigen of the SOW fraction which is capable of eliciting both humoral and cellular responses in infected patients. The SOWgp contributes to a bias in the Th1 versus Th2 pathways of immune response during the course of C. immitis infection.

### EXAMPLE

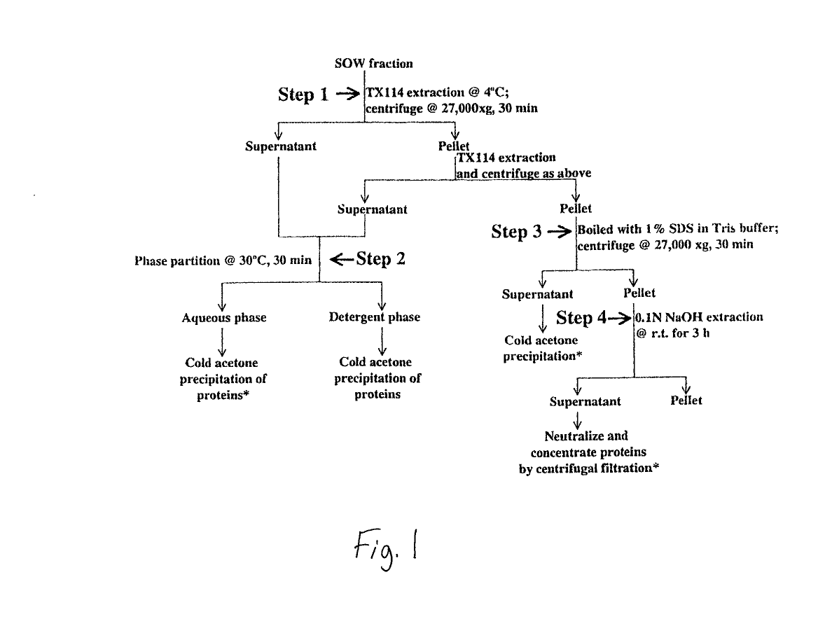

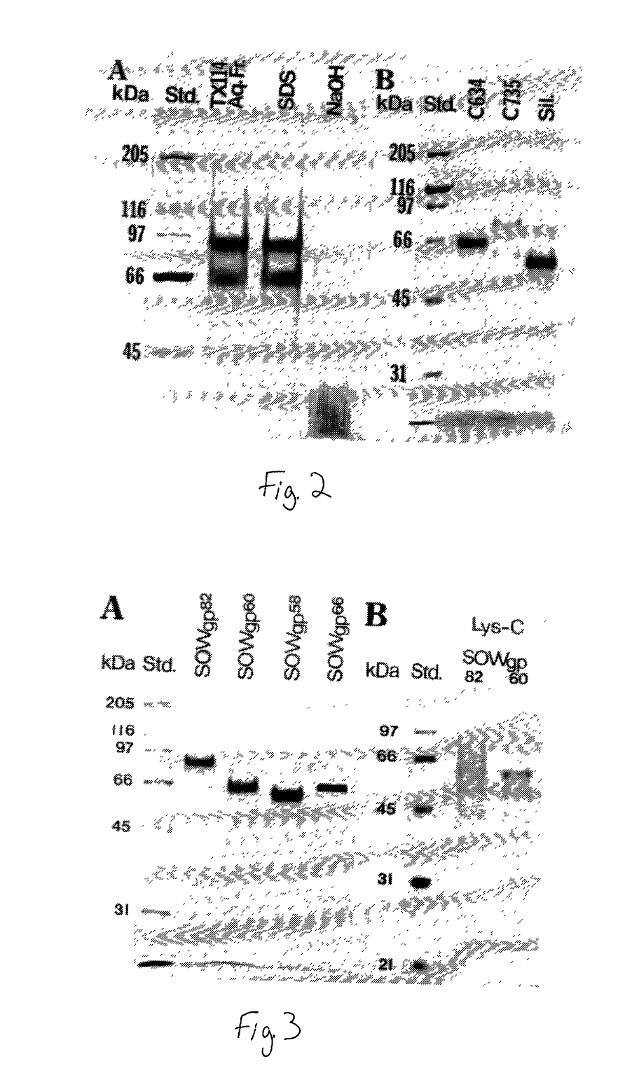

Major glycoprotein components of the crude SOW fraction. The SDS-polyacrylamide gel in FIG. 2A shows the silver-stained polypeptide components of the parasitic cell wall fraction of isolate C735. A comparison of electrophoretic separations of the aqueous and detergent phases of the TX114 extract of SOW from this isolate revealed that two stained bands with molecular sizes of 82 and 60 kDa were visible only in the aqueous phase. SDS extraction of the residual pellet after TX114 treatment released additional polypeptides with the same molecular sizes. No additional protein components were visible by SDS-PAGE after extraction of the insoluble wall fraction with 0.1 N NaOH (FIG. 1). Results of this same protein extraction procedure using SOW preparations obtained from isolates C634 and Silveira are shown in FIG. 2B and compared to results of isolate C735. The aqueous phase of the TX114 extract of C634 showed a prominent band with molecular size of 66 kDa, while the Silveira isolate revealed a single 58-kDa band.

The four protein bands of the three C. immitis isolates were electroeluted and applied to separate lanes of an SDS-10% polyacrylamide gel (FIG. 3A). After electrotransfer to an Immobilon-P membrane, each purified polypeptide was subjected to amino acid sequence analysis; the results are shown in Table 1. The N-terminal amino acid sequences of the four SOW components were identical. Each of the electroeluted fractions was also digested with either CNBr or Lys-C, the products were separated by SDS-PAGE (12% gel) and electrotransferred to membranes orchromatographically separated by RP-HPLC, and isolated peptides were subjected to N-terminal amino acid sequence analysis. The N-terminal sequences of internal peptides of SOWgps of the three isolates are shown in Table 1. Comparison of the Lys-C digests of the 82- and 60-kDa polypeptides of isolate C735 after SDS-PAGE separation is shown in FIG. 3B. Except for the upper bands of SOWgp82, with estimated molecular sizes of 82 and 77 kDa, the peptide fingerprints of the two SOW-extracted components of C735 were identical.

TABLE 1
Amino acid sequence analysis o the native glycoproteins and
peptide digests of SOWgps

| | Isolate/ | N-terminal amino acid sequence | |
|---|---|---|---|
| Protein | digestion | Native protein | Internal peptide |
| SOWgp82 | C735 | GATSHKEHSYXDTYG | |
| SOWgp60 | C735 | GATSHKEHSYXDTYG | |
| SOWgp66 | C634 | GATSHKEHSY | |
| SOWgp58 | Silveira | GATSHKEHSY | |
| SOWgp60 | C735/ | | TPKPXDCYCDCEDG |
| | Lys-C | | |
| SOWgp66 | C634/ | | KPEPPK(K/P)XD |
| | CNBr | | |
| SOWgp58 | Silveira/ | | YGDXDXYDGY |
| | Lys-C | | |

The polypeptides extracted from the SOW fraction of each isolate reacted with PAS stain (FIG. 4), indicating that they are glycosylated. Antiserum from patients with confirmed coccidioidal infection were shown in Western blots of the aqueous-soluble fraction to react with each of the glycoproteins (FIG. 4). No other bands were visible in this immunoblot of the TX114 extract. The 82-kDa component of C735 typically showed a more intense stain by Western blot analysis than the 60-kDa glycoprotein. Control sera failed to recognize the SOWgps.

An OG-solubilized fraction of SOW contained AgCS (29) and proline-rich Ag2 (PRAg2[20]). The immunoblot analysis was evaluated to determine whether the aqueous fraction of the TX114 extract also contained these two antigens. The SDS-PAGE separations of the aqueous fraction of SOW obtained from isolates C634, C735, and Silveira (FIG. 5A) were electrotransferred to two separate polyvinylidene difluoride membranes and reacted with antiserum raised against AgCS or PRAg2 (FIG. 5B and C, respectively). Purified rAgCS and rPRAg2 were also separated by SDS-PAGE and used as positive controls in the immunoblots. We were unable to detect ether AgCS or PRAg2 in the aqueous fractions of the TX114 extracts of SOW.

In summary, the three C. immitis isolates examined released a parasitic cell surface fraction (SOW) in vitro which was shown to contain a dominant glycoprotein (SOWgp) that ranged in molecular size from 82 to 58 kDa. The highest concentration of each isolate-specific glycoprotein during the extraction procedure was found in the aqueous fraction of an aqueous-detergent phase separation obtained during TX114 extraction of the crude SOW material (FIG. 1). The N-terminal amino acid sequences of all SOWgps were identical. Glycosylation of each polypeptide was indicated by positive PAS reaction, but the light stain suggested that the level of glycosylation was low. Each SOWgp was recognized by sera from patients with confirmed coccidioidal infection, and no other immuno-reactive components of the aqueous fraction of SOW were detected by immunoblot analyses. Patient antibody reactivity with the SOWgps that the glycoproteins are expressed in vivo and presented to the host during the parasitic cycle. This last feature of the SOWgps is further examined below.

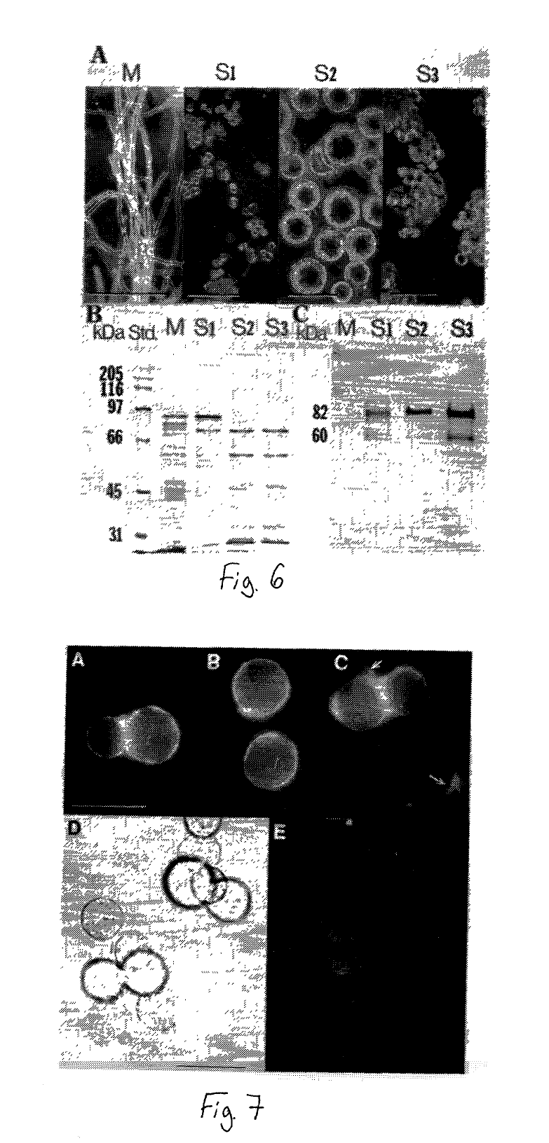

Parasitic phase-specific expression of SOWgps. Total homogenates of nonsporulating mycelia and parasitic cells of isolate C735 were obtained from late-log-phase saprobic cultures grown for 4 days and from parasitic-phase cultures grown for 1.5 to 5 days. The latter (FIG. 6A) represent different stages of spherule development (9). The total protein content of each homogenate that was applied to the SDS-PAGE gel in FIG. 6B was equilibrated. Western blot analysis of the protein separations was conducted with antiserum raised in mice against the purified SOWgp58 of the Silveira strain. The Western blot in FIG. 6C shows that the antiserum reacted with two components of each of the parasitic cell homogenates (molecular sizes of 82 and 60 kDa) but did not react with any of the gel-separated components of the mycelial phase. The two gel bands detected in parasitic cell homogenates are the same molecular sizes as the SOWgp components of the C735 isolate preparation shown in FIG. 2A. Since equal amounts of total protein were applied to lanes S1 to S3 in FIG. 6C, the higher intensity of the 82-kDa band visible in S3 indicated a higher percentage of this glyco-protein in the endosporulating spherule homogenate compared to the S1 (spherule initials) and S2 (segmented spherules) preparations.

Additional evidence that expression of SOWgp is phase specific is provided by results of immunolocalization studies. The same antiserum as noted above was incubated with vegetative hyphae and arthroconidia of the Silveira isolate obtained directly from GYE plate cultures. After incubation with the secondary antibody-FITC conjugate, the washed hyphal and arthroconidial samples failed to show any fluorescence. On the other hand, parasitic cells grown in vitro and reacted with the same primary and secondary antibodies showed a range of fluorescence intensity (FIG. 7A to C). Larger mature spherules appeared to bind higher amounts of primary antibody than young spherules (FIG. 7A). Moreover, a discontinuous pattern of fluorescence was revealed at the parasitic cell surface (FIG. 7B), and the SOW released into the media from the cell surface also reacted with the anti-SOWgp antibody (FIG. 7C). In vitro-grown spherules of the pathogen (FIG. 7D) incubated with the secondary antibody-FITC conjugate alone showed no significant level of fluorescence (FIG. 7E).

Patient humoral and cellular immunoreactivity with the SOWgps. Results of adsorption of patient and control serum samples to the purified SOWgp82 of isolate C735 are shown in FIG. 8. The test sera were derived from patients with coccidioidal infections, as well as from patients with confirmed B. dermatitides and Histoplasma capsulatum infections. Antibody adsorption to the SOWgp82 was examined by ELISA with a goat anti-human immunoglobulin G (heavy plus light chain) [IgG (H+L)] conjugated with peroxidase. The OD values are calculations of the means for triplicate assays.

Some cross-reactivity between the heterologous sera and SOWgp82 was evident. However, all patients with coccidioidal infections showed positive reaction with the test antigen, and the OD values for the majority of these sera were higher than the values for the heterologous serum samples. A comparison of ELISA data derived from the reactivity of these same coccidioidomycosis patient sera with SOWgp82 (from isolate C735) and SOWgp58 (from isolate Silveira) revealed that there is no significant difference in the OD values obtained despite the difference in molecular sizes of the two test antigens (Table 2). To further test whether the SOWgps from different isolates showed comparable reactivities with patient serum, we conducted an inhibition ELISA with a representative serum of a patient with confirmed coccidioidal infection (CF titer of 1:64 [Table 2]) and SOWgps from the Silveira (SOWgp58) and C735 (SOWgp82) isolates. The data shown in FIG. 9 demonstrate that the inhibition curves for reciprocal preincubation reactions with the patient serum are essentially superimposed.

A comparison of the CF antibody titers for this set of sera and the corresponding ELISA data are also presented in Table 2. A high CF titer is an indicator of dissemination of the C. immitis infection (30). There was no clear correlation between the CF titer and OD value for antibody reactivity with the SOWgp test antigens, at least for this sampling of patient sera.

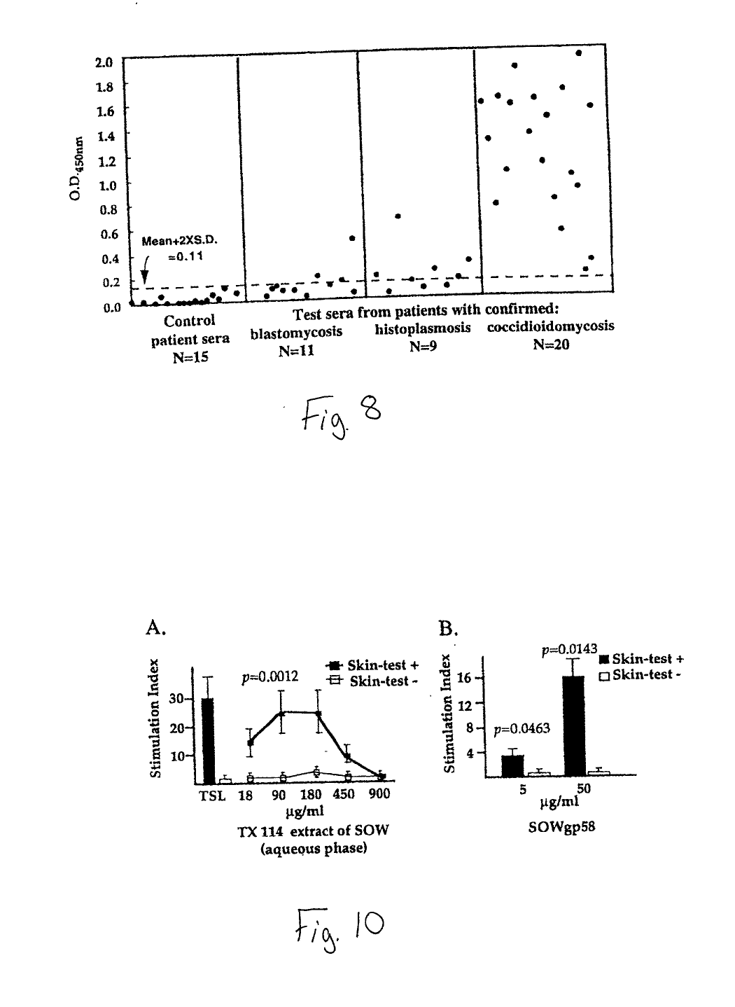

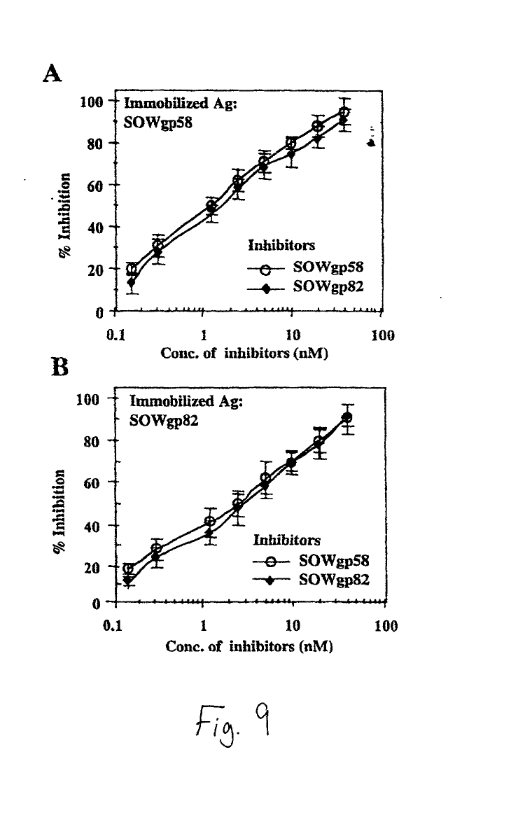

The immunoreactivity of the aqueous phase of the TX114 detergent-extracted SOW (from isolates C735 and Silveira) was compared to that of the purified SOWgp58 (from Silveira isolate) in patient PBMC proliferation assays (FIG. 10). When PBMC from eight immune donors (skin test positive for spherulin) were incubated with a range of concentrations of the TX114 extract of SOW, a dose-dependent increase in lymphocyte proliferation was recorded. Essentially no proliferation was observed when seven nonimmune (Skin test-negative) donors were tested (FIG. 10A). The peaks of proliferation in response to the aqueous-soluble fraction of the TX114 extract of SOW from cells from immune donors occurred at concentrations of 90 and 180 μg/ml. The SI was significantly higher at both of these concentrations than the SI for cells from nonimmune donors (P=0.001). At concentrations of TX114-extracted SOW above 180 μg/ml, a decrease in proliferation of PBMC from immune donors was observed. The purified SOWgp58 also stimulated PBMC proliferation of immune but not nonimmune donors (FIG. 10B). A comparison of PBMC from five skin test-positive and four skin test-negative volunteers showed that maximum proliferation of the former occurred when the cells were incubated with the SOWgp58 at a concentration of 50/μg/ml of cell culture medium. Results of a statistical analysis of proliferation data for assays conducted with the purified antigen at 5 and 50 μg/ml indicated that the SI for immune donors was significantly higher than the SI for nonimmune donors (P=0.014). The endotoxin content of the aqueous-soluble fraction of SOW and the purified SOWgp58 was less than 0.5 IU/μg of protein.

Culture supernatants harvested from PBMC incubated with the aqueous-soluble fraction of the TX114 extract of SOW (90 μg/ml) for 24 h were assayed by the ELISA for the presence of the cytokines INF-γ and IL-10. The mean concentration (±the standard error of the mean) of INF-γ for PBMC from seven immune donors was 263.3±94.1 pg/ml, compared to 19.4±11.1 pg/ml for five nonimmune donors (P=0.027). On the other hand, the mean concentration of IL-10 from the same set of immune donors was 2.0±0.8 pg/ml, not statistically different from the concentration of IL-10 (0.7±0.3 pg/ml) obtained for the corresponding set of nonimmune donors (P=0.368).

TABLE 2
Comparison of results of immunodiffusion-CF tests and
ELISAs of sera from coccidioidomycosis patients

| Patient | Antibody | OD of ELISA with the purified SOWgp^{b} | |
|---|---|---|---|
| serum no. | titer^{a} | SOWgp82 (C735) | SOWgp58 (Silveira) |
| 1 | 512 | 1.30 ± 0.09 | 1.48 ± 0.02 |
| 2 | 256 | 1.64 ± 0.03 | 1.53 ± 0.02 |
| 10 | 256 | 1.48 ± 0.05 | 1.51 ± 0.03 |
| 16 | 128 | 1.97 ± 0.04 | 1.78 ± 0.05 |
| 18 | 128 | 1.60 ± 0.05 | 1.91 ± 0.02 |
| 4 | 64 | 1.02 ± 0.03 | 0.97 ± 0.03 |
| 6 | 64 | 1.88 ± 0.07 | 1.69 ± 0.04 |
| 12 | 64 | 0.52 ± 0.03 | 0.51 ± 0.05 |
| 19 | 64 | 1.55 ± 0.16 | 1.88 ± 0.01 |
| 7 | 32 | 1.35 ± 0.13 | 1.38 ± 0.03 |
| 13 | 32 | 1.70 ± 0.05 | 1.46 ± 0.03 |
| 15 | 32 | 0.87 ± 0.06 | 0.84 ± 0.02 |
| 3 | 16 | 0.74 ± 0.03 | 0.55 ± 0.04 |
| 5 | 16 | 1.59 ± 0.06 | 1.13 ± 0.03 |
| 8 | 16 | 1.63 ± 0.04 | 1.53 ± 0.04 |
| 14 | 16 | 0.98 ± 0.01 | 0.86 ± 0.02 |
| 17 | 8 | 0.28 ± 0.01 | 0.56 ± 0.02 |
| 20 | 8 | 0.19 ± 0.06 | 0.47 ± 0.04 |
| 9 | 4 | 1.08 ± 0.03 | 1.59 ± 0.01 |
| 11 | 4 | 0.78 ± 0.04 | 0.59 ± 0.05 |
| ^{a}CF antibody titer for each serum sample, obtained by using the immunodiffusion-CF test (16). | | | |
| ^{b}Reported as the mean ± standard deviation of triplicate assays. SOWgps were used in the ELISA at a concentration of 10 ng in 100 μl of PBS per well of microdilution plate. The secondary antibody was a goat anti-human IgG (H + L)-peroxidase conjugate. Mean OD values for reactivity of test sera with SOWgp82 are plotted in FIG. 8. The correlation coefficient of the OD₄₅₀ values with SOWgp82 or SOWgp58 as the test antigen was 0.883 (P = 0.001), as calculated | | | |
| # with the aid of the SPSS statistical analysis program. | | | |

### MATERIALS AND METHODS

Cultivation of C. immitis and isolation of the SOW fraction. C. immitis C634, C735, and Silveira were grown as the saprobic phase on GYE agar plates (30 days, 25° C.) for production of arthroconidia or in GYE liquid culture (4 days) for vegetative mycelium production. Arthroconidia harvested from plate cultures were used to inoculate modified liquid Converse medium for growth of the parasitic phase as previously described (24). Spherule development was monitored by light microscopy. The SOW fraction released from the surface of spherules was isolated from the culture media by centrifugation, washed with distilled water, and lyophilized as reported elsewhere (8).

Extraction of the SOWgp fraction. Approximately 100 mg of lyophilized SOW was suspended in 5 ml of 1% Triton X-114 (TX114; Sigma Chemical Co., St. Louis, Mo.) in filtered distilled water containing 50 mM Tris-HCl (pH 6.8), 100 mM NaCl, and complete protease inhibitor cocktail at the concentration recommended by the manufacturer (Boehringer Mannheim, Indianapolis, Ind.). The sample in the TX114 extract buffer was incubated for 1 h at 4° C. with vigorous shaking and then centrifuged for 30 min (4° C.) at 27,000×g. The insoluble pellet was extracted again as described above; the supernatants were combined and then incubated at 30° C. for 30 min without agitation to obtain an aqueous-detergent phase separation (32). Each phase was transferred to separate centrifuge tubes, and the protein components of the aqueous and detergent phases were precipitated with five times the volume of ice-cold, absolute acetone overnight. The acetone-precipitated proteins were washed once with 80% acetone, resuspended in ultrapure MilliQ water (Millipore Corp., Bedford, Mass.), and then precipitated again in absolute ethanol to remove lipids and detergent. The protein fraction was resuspended in MilliQ water and separated by sodium dodecyl sulfate-polyacrylamide gel electrophoresis (SDS-PAGE) as previously described (41) except that the running buffer contained Tris (5.8 g/liter), glycine (28.8 g/liter), and SDS (1%). The protein concentration loaded on the gel was determined with a Bio-Rad (Hercules, Calif.) protein assay kit. The gels were stained with Coomassie blue (Sigma) or silver (Bio-Rad). The insoluble pellet derived from TX114 extraction of the crude wall material at 4° C. was further extracted by boiling for 5 min in 1% SDS solubilized in 20 mM Tris-HCl (pH 6.8) plus 1% β-mercaptoethanol. The insoluble material was pelleted, and the proteins in the supernatant were precipitated with acetone and separated by SDS-PAGE as described above. The protein concentration of the sample applied to the gel was adjusted to equal that of the TX114-derived extracts. Finally, the insoluble pellet derived from SDS extraction was suspended in 0.1 N NaOH for 3 h at room temperature. The supernatant obtained from this final extraction step was neutralized with 2 N HCl, the protein components were concentrated by passage through a Millipore Ultrafree-4 centrifugal filter unit with a 10-kDa cutoff (Millipore Corp.), and the entire retentate was subjected to SDS-PAGE. A summary of this four-step extraction procedure is presented in FIG. 1.

Protein purification and amino acid sequence analysis. The acetone-precipitated protein fraction of the aqueous phase obtained after TX114 extraction of SOW (FIG. 1) was subjected to preparative SDS-PAGE with the same running buffer as described above. The polypeptides of each C. immitis isolate were visualized in the gel with copper stain (Bio-Rad). The prominent bands were excised, destained, and electroeluted (10) in half-strength SDS-PAGE running buffer. The eluates were separately concentrated to 0.4 ml by passing the sample through a centrifugal filter followed by washes with MilliQ water. The isolated proteins were precipitated with acetone, washed once with 80% acetone, and finally washed with 100% ethanol as described above. The protein precipitate of each isolate (C634, C735, and Silveira) was resuspended in phosphate-buffered saline (PBS), subjected to SDS-PAGE (10% polyacrylamide), and electrotransferred to an Immobilon-P membrane (Millipore) as previously reported (29) except that the transfer solution contained 10% methanol, 0.2 M glycine, and unbuffered 20 mM Tris. The location of the band(s) of each isolate was visualized by Coomassie blue staining. The membrane-bound protein was excised, and its N-terminal amino acid sequence was determined with a Perkin-Elmer-Applied Biosystems model 428 amino acid analyzer. For internal amino acid sequence analyses, the electroeluted polypeptides were subjected to either CNBr (Sigma) or endopeptidase Lys-C (Promega Corp., Madison, Wis.) digestion. The digests were either separated by SDS-PAGE (12% polyacrylamide) and transferred to membranes as described above or fractionated by C₁₈ reverse-phase high-pressure liquid chromatography (RP-HPLC) as previously described (41). The isolated peptides were finally subjected to N-terminal amino sequence analysis as described above. The two polypeptides of isolate C735 (82 and 60 kDa) were electroeluted and separately incubated with Lys-C for 60 min as reported elsewhere (29). The peptide fingerprints of these digestions were compared by SDS-PAGE (12% gel).

Glycosylation analysis. The isolated polypeptide components of the crude SOW fraction were tested for the presence of sugar residues. The acetone-precipitated proteins of the aqueous phase of the TX114 extract (FIG. 1) of each SOW isolate were separated by SDS-PAGE (10% polyacrylamide), stained with periodic acid-Schiff (PAS) reagent, and then destained with 5% acetic acid as described elsewhere (13) for qualitative analysis of glycosylation.

Western blot analysis using patient sera. Western blots were prepared essentially as described previously (11), using sera from patients with confirmed, active coccidioidomycosis or rabbit and goat antisera raised against purified native antigen CS (AgCS[29]) and purified native proline-rich antigen 2 (PRAg2[20]), respectively. The solution used for electrotransfer of SDS-PAGE separations of SOWgp, AgCS, and PRAg2 preparations to polyvinylidene difluoride membranes (Millipore) contained 0.2 M glycine, unbuffered 20 mM Tris, and 10% methanol. Recombinant AgCS (rAgCS) (29) and recombinant PRAg2 (rPRAg2) (provided by J. N. Galgiani, Tucson, Ariz.) were used as positive controls.

Production of murine antisera. BALB/c mice were immunized subcutaneously with 10 μg of the purified SOWgp (SOWgp58) from the Silveira isolate solubilized in 50 μl of complete Freund's adjuvant (Sigma) was added. The mice were boosted twice at 2-week intervals with the same amount of immunogen plus incomplete Freund's adjuvant. Serum samples were tested for antibody specificity by Western blot analysis after the second and third boosts. Mice were sacrificed and exsanguinated by cardiac puncture at 2 weeks after the third boost.

Expression and immunolocalization of SOWgp. Antiserum raised against the purified SOWgp (Silveira) was used to examine expression of the glycoprotein during growth of the saprobic and parasitic phases of each isolate. The mycelial phase was harvested after growth in GYE medium for 4 days. Parasitic-phase cultures were grown in Converse medium and harvested after 1.5, 3, or 5 days. Total homogenates of the saprobic and parasitic phases (approximately 0.1 ml of each) were obtained by glass bead homogenization using a Mini-Beadbeater (Biospec Products, Bartlesville, Okla.) at 4° C. in the presence of the TX114 extract buffer. The insoluble material was pelleted by centrifugation (27,000×g, 20 min, 4° C.) and reextracted with the TX 114 buffer as described above. The supernatants were combined, and the protein concentration was estimated by using a Bio-Rad protein assay kit. Approximately 20 μl of each sample, which contained 30 μg of protein, was separated by SDS-PAGE (10% polyacrylamide) and electrotransferred to nitrocellulose membranes for Western blot analysis using the murine anti-SOWgp58 antiserum. The preimmune and test sera were diluted 1:500 in PBS containing 0.5% Tween 20 prior to reaction with the membrane. Duplicate gels were stained with Coomassie blue to confirm protein separation and that equal amounts of protein were added to each lane.

Freshly isolated arthroconidia and vegetative mycelia (Silveira isolate) grown on GYE agar for approximately 30 days, and spherules grown in Converse liquid medium for 3 days, were reacted with either mouse preimmune or anti-SOWgp58 serum at a 1:200 dilution in PBS. The cells were then washed with PBS and incubated with goat anti-mouse immunoglobulin conjugated with fluorescein isothiocyanate (FITC; Sigma) at room temperature for 15 min. Specimens were mounted on glass slides and examined by fluorescence microscopy as described elsewhere (8).

ELISA. The indirect ELISA was performed with a screening kit (Kirkegaard & Perry Laboratories, Gaithersburg, Md.) as previously described (7). Sera from 20 patients with confirmed coccidioidal infection and complement fixation (CF) antibody titers to C. immitis antigen (16) that ranged from 1:4 to 1:512 were tested fro reactivity with the purified SOWgp from isolates C735 and Silveira. Sera from patients with confirmed blastomycosis (11 samples) and histoplasmosis (9 samples) and control sera from 15 patients with no systemic or pulmonary mycoses were also tested. All serum samples were diluted 1:2,000 in blocking solution (11). Optimal antigen dilutions were determined by block titration with a representative set of sera from patients with coccidioidomycosis which showed the full range of CF titers. The final concentration of antigen applied to the microtiter wells was 10 ng in 100 μl of PBS. Test sera with an absorbance at 450 nm that was higher than the mean optical density (OD) value of the control sera plus two times the standard deviation were considered to be positive. Assays with sera in the absence of antigen and with antigen in the absence of sera served as controls. All sera were tested in triplicate wells, and the average OD value was presented. Correlation coefficients were calculated for the relationship of serum immunoreactivity between SOWgps from the two isolates (C735 and Silveira), using the SPSS statistical analysis program (version 6.1.1; Statistical Product and Service Solutions, Inc., Chicago, Ill.).

The inhibition ELISA was performed with patient serum 6 (see Table 2) and purified SOWgps from isolate Silveira (SOWgp58) and isolate C735 (SOWgp82). The representative patient serum (CF titer of 1:4) was preincubated with different concentrations of SOWgp or bovine serum albumin (BSA; Sigma). The concentrations of antigens prepared in PBS and used for preincubation were 0.16, 0.31, 0.63, 1.25, 2.50, 5.00, 10.00, 20.00, and 40.00 nM. The final dilution of the patient serum in the reaction mixture was 1:2,000. The reaction mixture was incubated for 30 min at room temperature. An aliquot (100 μl) of the patient serum plus antigen after preincubation was added to each well of a microtiter plate precoated with the homologous or heterologous antigen. The final concentration of antigen (SOWgp58 or SOWgp82) which coated the microtiter wells was 10 ng in 100 μl of PBS. The assays were performed as described above. Percent inhibition was calculated on the basis of the OD value for the patient serum preincubated with BSA minus the OD value for the patient serum preincubated with SOWgp divided by the OD value for the patient serum incubated by BSA×100.

Human PBMC proliferation assay. A peripheral blood monocytic cells (PBMC) proliferation assay was performed as previously described (2). Donors consisted of healthy individuals without any evidence of active coccidioidomycosis. All donors underwent skin testing with spherulin, a commercially available (ALK Laboratories, Berkeley, Calif.) parasitic-phase antigen complex used for measuring immunological reactivity (15). Donors were categorized as immune if they expressed delayed-type dermal hypersensitivity to the C. immitis antigen preparation and as nonimmune if they did not. All work with human donors was approved by the Human Subjects Committee of the University of Arizona. PBMC were isolated from heparinized venous blood of the donors by using a Ficoll-Hypaque separation column (Pharmacia, Piscataway, N.J.). Subsequently, 5×10⁵ viable PBMC were added to separate wells of flat-bottom 96-well plates (Corning Glass Works, Corning, N.Y.) in RPMI 1640 (GIBCO, Grand Island, N.Y.) containing 10% heat-inactivated, pooled human AB serum (GIBCO). Antigen (aqueous phase of the TX114-extracted SOW or purified SOWgp from the Silveira isolate) was added to test wells at concentrations of 5 to 900 μg/ml of cell culture medium, and the plate was incubated for 5 days at 37° C. in 95% air-5% CO₂. The control antigen, a toluene spherule lysate which has been shown to be a potent stimulator of PBMC response (2), was added to test wells at a concentration of 100 μg/ml. [³H]thymidine (0.5 μCi/ml; NEN, Boston, Mass.) was added to each well, and after an additional 18 h of incubation, cells were harvested onto glass filter paper, which was analyzed by scintillation spectrometry. The endotoxin content of each antigenic preparation was measured with a Limulus amebocyte lysate chromogenic kit (BioWhittaker, Walkersville, Md.). Results of the PBMC proliferation assay are expressed as a stimulation index (SI), calculated as the counts per min of wells containing the antigen divided by the counts per minute of control wells without antigen. The Mann-Whitney U test was used for a statistical comparison of the stimulation index for proliferation assays using PBMC of immune and nonimmune donors.

Cytokine assay of PBMC supernatants. The assay was performed as previously described (2), using precoated ELISA plates for determination of the amounts of specific cytokines. PBMC were incubated for 24 h in flat-bottom wells of 96-well plates as described above. Supernatant fluid was then aspirated from groups of 8 to 12 wells of test or control samples, separately pooled, and immediately frozen at −70° C. Quantitative assays of gamma interferon (INF-γ) and interleukin-10 (IL-10) concentrations in the cell supernatants were performed by ELISA according to the protocol of the manufacturer of the assay kit (Biosource International, Camarillo, Calif.).

The following references which are cited herein are expressly incorporated by reference.

1. Almeida, S. R., C. S. Unterkircher, and Z. P. Camargo. 1998. Involvement of the major glycoprotein (gp43) of Paracoccidioides brasiliensis in attachment to macrophages. Med. Mycol. 36:405-411.

2. Ampel, N. M., and L. Christian. 1997. In vitro modulation of proliferation and cytokine production by human peripheral blood mononuclear cells from subjects with various forms of coccidioidomycosis. Infect. Immun. 65:4483-4487.

3. Bernard, F., M. A. Hong, G. M. Negro, L. Batista, M. A. Shikanai-Yasuda, and A. J. Duarte. 1996. Antigen-specific immunosuppression in paracoccidioidomycosis. Am. J. Trop. Med. Hyg. 54:7-12.

4. Benard, G., M. J. Mendes-Giannini, M. Juvenale, E. J. Miranda, and A. J. Duarte. 1997. Immunosuppression in paracoccidioidomycosis: T cell hypo-responsiveness to two Paracoccidioides brasiliensis glycoproteins that elicit strong humoral immune response. J. Infect. Dis. 175:1263-1267.

5. Brandhorst, T. T., M. Wuthrich, T. Warner, and B. Klein. 1999. Targeted gene disruption reveals an adhesin indispensable for pathogenicity in Blastomyces dermatitidis. J. Exp. Med. 189:1207-1216.

6. Chandler, F. W., W. Kaplan, and L. Ajello. 1980. Histopathology of mycotic diseases. Year Book Medical Publishers, Inc., Chicago, Ill.

7. Cole, G. T., D. Kruse, S. Zhu, K. R. Seshan, and R. W. Wheat. 1990. Composition, serologic reactivity, and immunolocalization of a 1 20-kilodalton tube precipitin antigen of Coccidioides immitis. Infect. Immun. 58:179-188.

8. Cole, G. T., K. R. Seshan, M. Franco, E. Bukownik, S. H. Sun, and V. M. Hearn. 1988. Isolation and morphology of an immunoreactive outer wall fraction, produced by spherules of coccidioides immitis. Infect. Immun. 56:2686-2694.

9. Cole, G. T., and S. H. Sun. 1985. Arthroconidium-spherule-endospore transformation in Coccidioides immitis, p. 281-333. In P. J. Szaniszlo (ed.), Fungal dimorphism. Plenum Publishing Corp., New York, N.Y.

10. Cole, G. T., S. Zhu, S. Pan, L. Yuan, D. Kruse, and S. H. Sun. 1989. Isolation of antigens with proteolytic activity from Coccidioides immitis. Infect. Immun. 57:1524-1534.

11. Cole, G. T., T. N. Kirkland, M. Franco, S. Zhu, L. Yuan, S. H. Sun, and V. M. Hearn. 1988. Immunoreactivity of a surface wall fraction produced by spherules of Coccidioides immitis. Infect. Immun. 56:2695-2701.

12. Corry, D. B. , N. M. Ampel, L. Christian, R. M. Locksley, and J. N. Galgiani. 1996. Cytokine production by peripheral blood mononuclear cells in human coccidioidomycosis. J. Infect. Dis. 174:440-443.

13. Dubray, G., and G. Bezard. 1982. A high sensitive periodic acid-silver stain for the 1,2-diol group of glycoproteins and polysaccharides in polyacrylamide gels. Anal. Biochem. 119:325-329.

14. Frey, C. L., and D. J. Drutz. 1986. Influence of fungal surface components on the interaction of Coccidioides immitis with polymorphonuclear neutrophils. J. Infect. Dis. 153:933-943.

15. Huppert, M. 1983. Antigens used for measuring immunological reactivity, p. 219-302. In D. Howard (ed.), Fungi pathogenic for humans and animals, part B: pathogenicity and detection. Marcel Dekker, Inc., New York, N.Y.

16. Huppert, M., and J. W. Bailey. 1965. The use of immunodiffusion tests in coccidioidomycosis. I. The accuracy and reproducibility of the immunodiffusion test which correlates with complement fixation. Am. J. Clin. Pathol. 44:364-368.

17. Janeway, D. A., P. Travers, M. Walport, and J. D. Capra. 1999. Immunobiology: the immune system in health and disease, 4th ed. Current Biology Publications, London, United Kingdom.

18. Johnson, S. M., C. S. Zimmerman, and D. Pappagianis. 1992. The coccidioidal complement fixation and immunodiffusion-complement fixation antigen is a chitinase. Infect. Immun. 60:2588-2592.

19. Kirkland, T. N., P. W. Thomas, F. Finley, and G. T. Cole. 1998. Immunogenicity of a 48-kilodalton recombinant T-cell-reactive protein of Coccidioides immitis. Infect. Immun. 66:424-431.

20. Kirkland, T. N., F. Finley, K. l. Orsborn, and J. N. Galgiani. 1998. Evaluation of the proline-rich antigen of Coccidioides immitis as a vaccine candidate in mice. Infect. Immun . 66:3519-3522.

21. Klein, B. S., S. Chaturvedi, L. H. Hogan, J. M. Jones, and S. L. Newman. 1994. Altered expression of the surface protein WI-1 in genetically related strains of Blastomyces dermatitidis that differ in virulence regulates recognition of yeasts by human macrophages. Infect. Immun. 62:3536-3542.

22. Klein, B. S., and S. L. Newman. 1996. Role of cell-surface molecules of Blastomyces dermatitidis in host-pathogen interactions. Trends Microbiol. 4:246-251.

23. Kruse, D., and G. T. Cole. 1992. A seroreactive 120-kilodalton,8-1,3-glucanase of Coccidioides immitis which may participate in spherule morpho-genesis. Infect. Immun. 60:4350-4363.

24. Levine, H. B. 1961. Purification of the spherule-endospore phase of Coccidioides immitis. Sabouraudia 1:112-115.

25. Magee, D. M., and R. A. Cox. 1995. Roles of gamma interferon and interleukin-4 in genetically determined resistance to Coccidioides immitis. Infect. Immun. 63:3514-3519.

26. Magee, D. M., and R. A. Cox. 1996. Interleukin-12 regulation of host defenses against Coccidioides immitis. Infect. Immun. 64:3609-3613.

27. Man, N. K., A. Degrement, J. C. Darbord, M. Collet, and P. Vaillant. 1998. Evidence of a bacterial biofilm in tubing from the hydraulic pathway of a hemodialysis system. Artif. Organs 22:596-600.

28. Orsborn, K. I., and J. N. Galgiani. 1998. Detecting serum antibodies to a purified recombinant proline-rich antigen of Coccidioides immitis in patients with coccidioidomycosis. Clin. Infect. Dis. 27:1475-1478.

29. Pan, S., and G. T. Cole. 1995. Molecular and biochemical characterization of a Coccidioides immitis-specific antigen. Infect. Immun. 63:3994-4002.

30. Pappagianis, D., and B. L. Zimmer. 1990. Serology of coccidioidomycosis. Clin. Microbiol. Rev. 3:247-268.

31. Pishko, E. J., T. N. Kirkland, and G. T. Cole. 1995. Isolation and characterization of two chitinase-encoding genes (cts1, cts2) from the fungus Coccidioides immitis. Gene 167:173-177.

32. Pryde, J. G. 1997. Partitioning of proteins in TritonX-114. Methods Mol. Biol. 88:23-33.

33. Saraiva, E. C. O., A. Altemani, M. F. Franco, C. S. Unterkircher, and Z. P. Camargo. 1996. Paracoccidioides brasiliensis-gp43 used in paracoccidioidin. J. Med. Vet. Mycol. 34:155-161.

34. Stark, R. M., G. J. Gerwig, R. S. Pitman, L. F. Potts, N. A. Williams, J. Greenman, I. P. Weinzweig, T. R. Hirst, and M. R. Millar. 1999. Biofilm formation by Helicobacter pylori. Lett. Appl. Microbiol. 28:121-126.

35. Sun, S. H., G. T. Cole, D. J. Durtz, and J. L. Harrison. 1986. Electron-microscopic observations of the Coccidioides immitis parasitic cycle in vivo. J. Med. Vet. Mycol. 24:183-192.

36. Tarbet, J. E., and A. M. Breslau. 1953. Histochemical investigation of the spherule of Coccidioides immitis in relation to host reaction. J. Infect. Dis. 72:183-190.

37. Vicentini, A. P., J.-L. Gesztesi, M. F. Franco, W. de Souza, J. Z. de Moraes, L. R. Travassos, and J. D. Lopes. 1994. Binding of Paracoccidioides brasiliensis to laminin through surface glycoprotein gp43 leads to enhancement of fungal pathogenesis. Infect. Immun. 62:1465-1469.

38. Wüthrich, M., W.-L. Chang, and B. S. Klein. 1998. Immunogenicity and protective efficacy of the WI-1 adhesion of Blastomyces dermatitidis. Infect. Immun. 66:5443-5449.

39. Yang, C., Y. Zhu, D. M. Magee, and R. A. Cox. 1996. Molecular cloning and characterization of the Coccidioides immitis complement fixation/chitinase antigen. Infect. Immun. 64:1992-1997.

40. Yuan, L, and G. T. Cole. 1987. Isolation and characterization of an extra-cellular proteinase of Coccidioides immitis. Infect. Immun. 55:1970-1978.

41. Yuan, L. and G. T. Cole. 1989. Characterization of a proteinase inhibitor isolated from the fungal pathogen Coccidioides immitis.

Biochem. J. 257:729-736.

We claim:

1. A recombinant spherical outer wall glycoprotein 58 (SOWgp58).

2. An antigen for serodiagnosis of coccidioidomycosis comprising a recombinant spherical outer wall glycoprotein.

3. The antigen of claim 2, wherein the glycoprotein comprises SOWgp58.

4. The antigen of claim 2, wherein the glycoprotein comprises an isolated DNA sequence substantially as shown in Sequence ID No. 1.

5. The antigen of claim 2, wherein the glycoprotein comprises an amino acid sequence substantially as shown in Seq. ID No. 2.

6. The antigen of claim 2, wherein the glycoprotein comprises an isolated DNA sequence substantially as shown in Seq. ID No. 3.

7. The antigen of claim 2, wherein the glycoprotein comprises an amino acid sequence substantially as shown in Seq. ID No. 4.

8. The antigen of claim 2, wherein the glycoprotein comprises an amino acid sequence substantially as shown in Seq. ID No. 5.

9. The antigen of claim 2, wherein the glycoprotein comprises an amino acid sequence substantially as shown in Seq. ID No. 6.

10. The antigen of claim 2, wherein the glycoprotein comprises an amino acid sequence substantially as shown in Seq. ID No. 7.

11. The antigen of claim 2, wherein the glycoprotein comprises an amino acid sequence substantially as shown in Seq. ID No. 8.

12. The antigen of claim 2, wherein the glycoprotein comprises an isolated DNA sequence substantially as shown in Seq. ID No. 9.

13. The antigen of claim 2, wherein the glycoprotein comprises an amino acid sequence substantially as shown in Seq. ID No. 10.

14. The antigen of claim 2, wherein the glycoprotein comprises an isolated DNA sequences substantially as shown in Seq. ID No. 11.

15. The antigen of claim 2, wherein the glycoprotein comprises an amino acid substantially as shown in Seq. ID No. 12

16. An isolated nucleic acid comprising a sequence that hybridizes under highly stringent conditions to a hybridization probe the nucleotide sequence of which consists of Seq. ID No. 1, or the complement of Seq. ID No. 1.

17. An isolated nucleic acid comprising a sequence that hybridizes under highly stringent conditions to a hybridization probe the nucleotide sequence of which consists of Seq. ID No. 3, or the complement of Seq. ID No. 3.

18. An isolated nucleic acid comprising a sequence that hybridizes under highly stringent conditions to a hybridization probe the nucleotide sequence of which consists of Seq. ID No. 9, or the complement of Seq. ID No. 9.

19. An isolated nucleic acid comprising a sequence that hybridizes under highly stringent conditions to a hybridization probe the nucleotide sequence of which consists of Seq. ID No. 11, or the complement of Seq. ID No. 11.

20. A purified polypeptide, the amino acid of which comprises Seq. ID No. 2 with 0 to 20 conservative amino acid substitutions.

21. A purified polypeptide, the amino acid of which comprises Seq. ID No. 4 with 0 to 20 conservative amino acid substitutions.

22. A purified polypeptide, the amino acid of which comprises Seq. ID No. 5 with 0 to 20 conservative amino acid substitutions.

23. A purified polypeptide, the amino acid of which comprises Seq. ID No. 6 with 0 to 20 conservative amino acid substitutions.

24. A purified polypeptide, the amino acid of which comprises Seq. ID No. 7 with 0 to 20 conservative amino acid substitutions.

25. A purified polypeptide, the amino acid of which comprises Seq. ID No. 8 with 0 to 20 conservative amino acid substitutions.

26. A purified polypeptide, the amino acid of which comprises Seq. ID No. 10 with 0 to 20 conservative amino acid substitutions.

27. A purified polypeptide, the amino acid of which comprises Seq. ID No. 12 with 0 to 20 conservative amino acid substitutions.

28. A method to elicit a proliferative response of monocytes of skin test positive animals, comprising introducing into the animal a composition comprising the purified antigen of claim 2.

29. A kit comprising a vessel or vessels containing the purified antigen of claim 2.

30. A method comprising:
a) obtaining a sample of nucleic acid from a subject; and
b) determining the identify of the nucleotide encoding the SOWgp58, SOWgp66 and/or SOW82 genes.

31. The method of claim 30, wherein the determining step comprises sequencing at least a portion of a nucleic acid molecule encoding at least one of said genes.

32. The method of claim 30, wherein the determining step comprises amplifying at least a portion of a nucleic acid molecule encoding at least one of said genes.
